(19) United States
(12) Patent Application Publication
(10) Pub. No.: US 2025/0213263 A1
(43) Pub. Date: Jul. 3, 2025

(54) DUPUYTREN'S CONTRACTURE TREATMENT DEVICE

(71) Applicant: Sonex Health, Inc., EAGAN, MN (US)

(72) Inventors: Darryl Eugene Barnes, Eagan, MN (US); Jay Smith, Inver Grove Heights, MN (US)

(73) Assignee: Sonex Health, Inc., EAGAN, MN (US)

(21) Appl. No.: 19/001,964

(22) Filed: Dec. 26, 2024

Related U.S. Application Data
(60) Provisional application No. 63/615,035, filed on Dec. 27, 2023.

(51) Int. Cl. A61B 17/32; A61B 17/00; A61B 90/00

(52) U.S. Cl. CPC A61B 17/32; A61B 90/36; A61B 2017/0046; A61B 2017/00743; A61B 2090/378

(57) ABSTRACT

A device for treating Dupuytren's contracture in a hand of a patient includes a handle, a shaft extending from the handle and sized to fit through a small incision on the hand and under a target tissue, a sharp distal tip on the shaft, a retrograde blade on the shaft, facing proximally toward the handle, and an opening in the shaft, adjacent the retrograde blade. In alternative embodiments, the retrograde blade faces either upward or downward relative to the shaft. Optionally, the treatment device may include a blade protector slidably disposed along the shaft from the handle to the retrograde blade and a slider on the handle, connected to the blade protector, to slide the blade protector along the shaft from a blade protecting position to a blade exposed position.

### CROSS-REFERENCE TO RELATED APPLICATIONS

This application claims the benefit of U.S. Provisional Patent Application No. 63/615,035, filed Dec. 27, 2023, the disclosure of which is incorporated herein by reference in its entirety for any and all purposes.

### TECHNICAL FILED

The present application relates generally to medical devices and methods. More specifically, the present application relates to a device and method for treating Dupuytren's contracture and possibly other soft tissue disorders of the hand, wrist, and/or other parts of the body.

### BACKGROUND

Dupuytren's contracture is a hand condition characterized by the thickening of tissue beneath the skin of the palm, causing the fingers to gradually curl into a bent position. The condition usually begins with small nodules or indentations in the palm, which can sometimes be painful. Over time, the nodules can form thick cords that pull the fingers into the palm. It can be challenging for a person suffering from Dupuytren's contracture to use the affected hand normally.

There is no known cure for Dupuytren's contracture, and many of the currently available treatments result in recurrence. Treatment options typically begin with conservative approaches, such as splinting and physical therapy. Needle aponeurotomy (also referred to as “fasciotomy,” since the tissue being penetrated beneath the skin is fascia) involves poking multiple, small holes through the skin and thickened tissue, to allow the tissue to stretch. Enzyme (collagenase) injections are considered a less invasive approach but often temporarily damage the skin and have significant complication rates. The common surgical approach, used if conservative measures are unsuccessful or the patient prefers surgery, is a fasciectomy, in which the thickened fascia (or “cord”) is removed.

Non-surgical approaches are recommended for early-stage cases of Dupuytren's contracture, while surgery is considered for advanced or severe contractures. The choice of treatment depends on factors such as the stage and progression of the condition, overall health, and individual preferences. Unfortunately, none of the approaches for treating Dupuytren's contracture acts as a permanent cure.

Ideally, minimally invasive or less invasive surgical devices and methods for treating Dupuytren's contracture would be available. Such devices and methods would ideally allow for treatment with less post-surgical pain, shorter and easier recovery, and less disability than with traditional surgical techniques.

### BRIEF DESCRIPTION

The present disclosure describes several examples of a device and method for treating Dupuytren's contracture. The device generally includes a handle, a shaft, and a blade formed in or attached to a distal portion of the shaft. A distal tip of the blade may be advanced through a small incision in the skin and then used, in some cases, to cut through subcutaneous tissue between the skin and the fascia that is affected by Dupuytren's contracture. The device may then be removed and reinserted under the fascia, and a blade on the device may be used to completely or partially remove an offending tissue that is causing the Dupuytren's contracture. One or more of the treatment steps may be repeated, as desired by the physician, to free up and/or remove tissue. In some examples, this tissue removal may be a complete or partial fasciectomy. One or more cords may be treated in any given embodiment, such as but not limited to a central cord. Generally, any tissue in the hand may be treated using examples of the device and method described herein.

### BRIEF DESCRIPTION OF THE DRAWINGS

FIG. 1 is a top view of a Dupuytren's contracture treatment device, according to one embodiment;

FIG. 2A is a close-up, top view of a distal portion of the treatment device of FIG. 1;

FIG. 2B is a close-up, side view of a distal portion of the treatment device of FIG. 1;

FIG. 2C is a close-up, front view of a distal portion of the treatment device of FIG. 1;

FIG. 3 is a palmar view of a hand, with an image of a distal portion of the Dupuytren's contracture treatment device overlayed on top of it, in an approximate position for a treatment, according to one embodiment, although the actual treatment is performed under the skin;

FIGS. 4A and 4B are perspective and side views, respectively, of a Dupuytren's contracture treatment device, according to an alternative embodiment; and

FIGS. 5A-5E are side, cross-sectional views of a patient's skin and underlying tissues on the hand, illustrating a method of treating Dupuytren's contracture using the treatment device of FIGS. 4A and 4B, according to one embodiment.

### DETAILED DESCRIPTION

The procedures described herein are typically, but not necessarily, performed under ultrasound guidance, with an ultrasound device positioned outside the hand and/or wrist to visualize structures inside the hand and/or wrist. Additionally, or alternatively, an ultrasound transducer may be incorporated into the surgical device itself, so that ultrasound visualization is provided directly inside the hand.

The embodiments of devices and methods described herein are examples only. Any given embodiment may include features of other described embodiments or may be altered or adapted for alternative uses, without departing from the scope of the invention.

In this application, the term “distal” generally means “close to or in a direction toward target tissue,” and the term “proximal” generally means “farther from or in a direction away from the target tissue” or “closer to the doctor or other person operating the device.” In other words, proximal and distal are relative terms. For example, when a user holds a treatment device and inserts one end of the treatment device into a patient to perform a treatment, the end of the device that is inserted into the patient will be referred to as the “distal end” of the device. The end of the device being held by the physician will be referred to as the “proximal end” of the device. Although these terms will be used consistently in this application, they should not be interpreted as limiting.

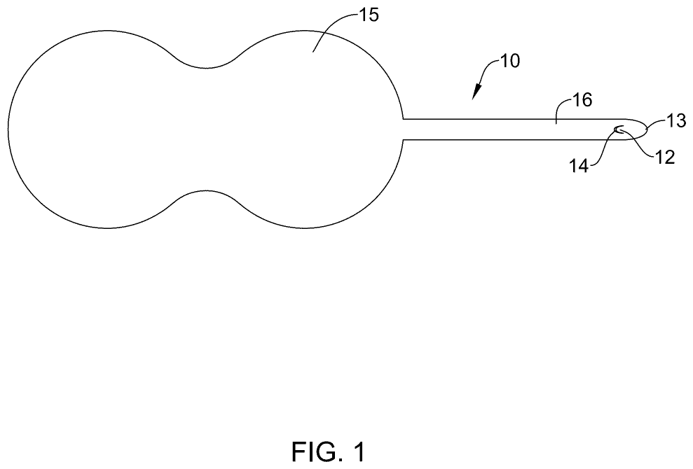

Referring now to FIG. 1, one embodiment of a Dupuytren's contracture treatment device 10 (or simply “treatment device 10”) is schematically illustrated in top view. The treatment device 10 is configured to perform a partial fasciectomy for Dupuytren's contractures. The treatment device comprises a handle 15, a shaft 16 extending from the handle 15, a retrograde cutting member 12 (or “blade”), a tissue opening 14 through which cut tissue can pass, and a sharp distal tip 13. In some embodiments, the treatment device 10 may be a single piece construction made of one piece and type of material, such as any suitable metal or polymer. Alternatively, the treatment device 10 may be made of multiple parts attached together. For example, the handle 15 may be a first piece, and the shaft 16 may be a second piece. In one embodiment, the handle 15 is plastic and the shaft 16 is metal. In some embodiments, the shaft 16 may have a tubular or semi-tubular cross-sectional shape.

The retrograde cutting member 12 faces proximally (“retrograde”) and downward, toward a bottom of the treatment device 10. The opening 14 is located on the upper/top surface of the shaft 16, just above and adjacent to the opening. Thus, in a Dupuytren's contracture treatment procedure, the distal tip 13 and retrograde cutting member 12 are advanced beyond (distal to) and above (superficial to) the tissue to be cut (also called a “cord” in this procedure). The physician then pulls back on the handle 15 to pull the retrograde cutting member 12 through the target tissue/cord and cut the tissue. The cut tissue then passes through the opening 14 in the top surface of the shaft 16.

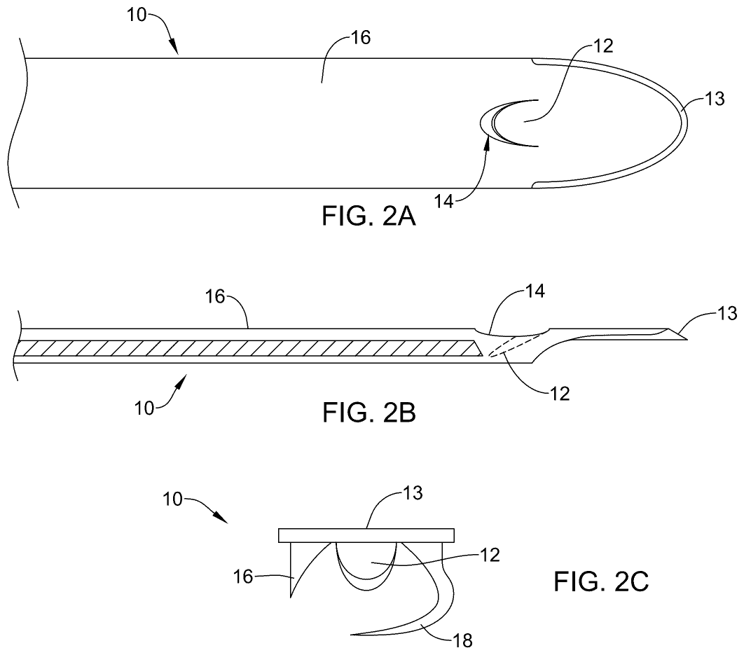

FIGS. 2A-2C are close-up views of a distal portion of the treatment device 10, in top, side, and front views, respectively. FIG. 2B shows an optional blade guard 17, disposed in the shaft 16, which protects the cutting member 12 and prevents it from cutting non-target tissue when being advanced into the patient's hand before the physician intends to cut tissue. FIG. 2C shows an optional tendon guard 18, which may be included in some embodiments to help protect tendons from being accidentally cut by the retrograde cutting member 12 during a Dupuytren's contracture treatment with the treatment device 10. Various embodiments may include one tendon guard 18 or two (one on each side). Alternatively, if the treatment device 10 includes the blade guard 17, the tendon guard 18 is not needed. The side view of FIG. 2B shows the retrograde cutting member 12 and the tissue opening 14 on the top of the treatment device 10.

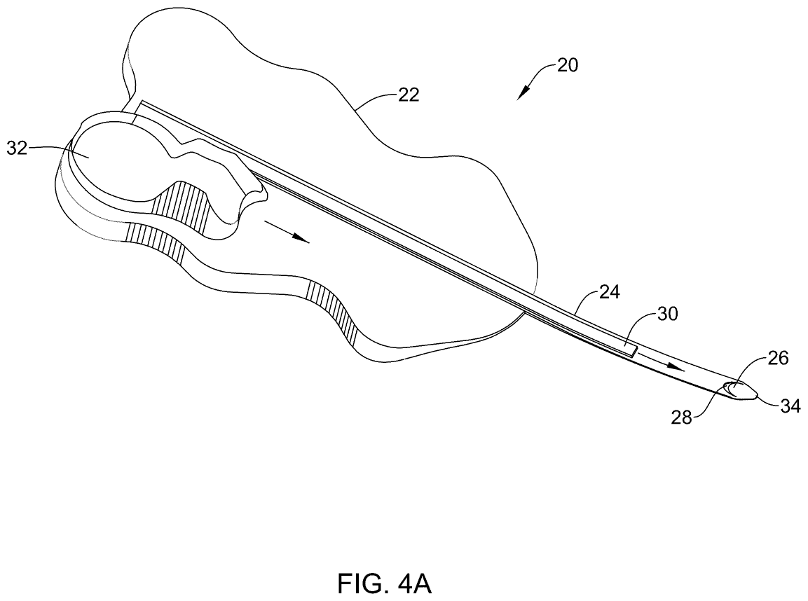

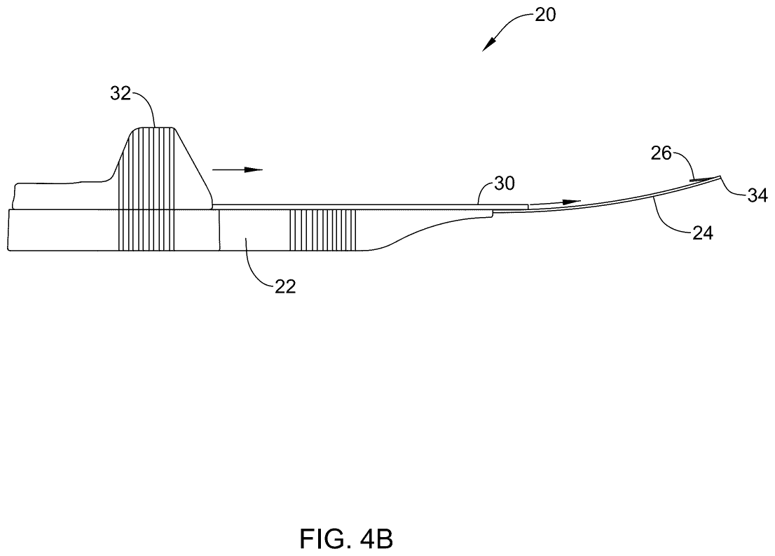

Referring now to FIGS. 4A and 4B, an alternative embodiment of a Dupuytren's contracture treatment device 20 (or “treatment device 20”) may include a handle 22, a shaft 24 extending from the handle 22 and including a sharp distal tip 34, a blade 26 (or “cutting member”) formed in the shaft 24, an opening 28 adjacent the blade 26, a blade protector 30, and a slider 32 positioned on the handle 22 and attached to the blade protector 30. As illustrated in FIG. 4B, in this embodiment, the blade 26 faces upward and proximally (retrograde), in contrast to the embodiment of FIGS. 2A-2C, where the retrograde cutting member 12 faces downward and proximally. In this embodiment, as well as in the previously described embodiment, the shaft 24 may have a gradual curve to assist with advancement and retraction of the shaft 24 from the hand during a procedure.

In some embodiments, the handle 22 and the shaft 24 may be one piece. Alternatively, the shaft 24 may be permanently attached to the handle. In yet other embodiments, the shaft 24 may be removable from the shaft 24 and may be swapped out for a different shaft by the user, i.e., a modular handle 22 and shaft 24 embodiment. The handle 22, shaft 24, blade 26, blade protector 30, and slider 32 may be made of any suitable, biocompatible material or combination of materials. For example, in one embodiment, the handle 22 and slider 32 are made of plastic, and the shaft 24, blade 26, and blade protector 30 are made of metal. In use, the slider 32 is advanced and retracted along the top of the handle 22 to advance and retract the blade protector 30. As described in further detail below, the blade protector 30 in the advanced position rests on or near the cutting surface of the blade 26 and prevents it from cutting, for example while the shaft 24 is being advanced into the hand. When the blade protector 30 is retracted, the blade protector 30 may free the cutting edge of the blade 26 to cut tissue.

In the embodiment shown, the shaft 24 and blade protector 30 are relatively flat. In one alternatively embodiment, the shaft 24 may be made from a hypotube and thus have a circular or oval cross-sectional shape. The blade 26 may be positioned or formed in the hypotube metal, and a sharp distal end of the hypotube shaft may be used to core a section out of the target tissue.

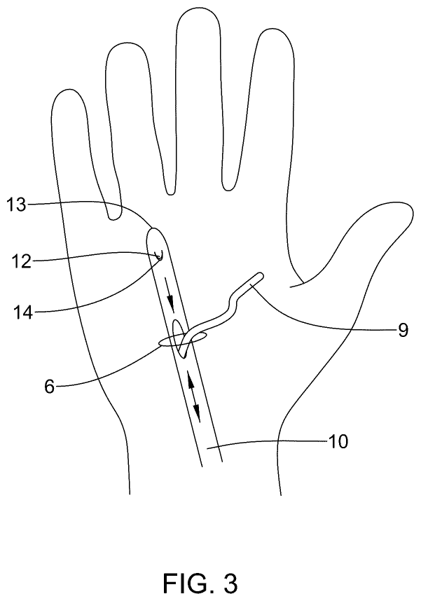

Referring now to FIG. 3, one embodiment of a method for treating Dupuytren's contracture using the Dupuytren's contracture treatment device 10 is illustrated in a simple drawing of a patient's hand. After typical surgical sterilization preparation of the skin and proper patient positioning, local anesthesia is injected in and around the surgical site, or a regional nerve block may be performed. Ultrasound may then be used to identify the central cord 6 (and/or other cord/fascia to be removed) and skin demarcating the neurovascular bundles on each side of the central cord 6. Next, the physician forms a proximal transverse incision, superficial to the central cord 6. Skin edges are then retracted to directly visualize the central cord 6.

Next, the physician advances the distal tip 13 of the treatment device 10 superficially to the central cord 6 and deep to the overlying skin. In a slight side-to-side motion, the treatment device 10 is advanced over the central cord 6, dissecting and separating the skin from the central cord 6. Once the distal tip 13 of the treatment device 10 has been advanced to its appropriate depth, the handle 15 is then firmly grasped and pulled back, cutting and isolating the central cord 6 with the retrograde cutting member 12. Once the distal tip 13 is at the level of the incision, it is removed, thus pulling out a cut segment 9 of the central cord 6. This cut segment 9 is then excised. The process may be repeated until the central cord 6 has been completely excised. This example of the method and device may be generally referred to as a “downward blade” method and device.

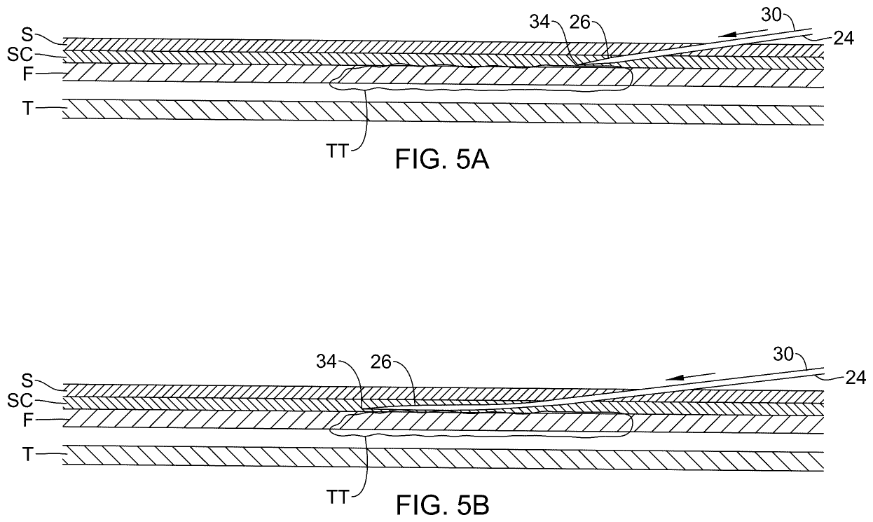

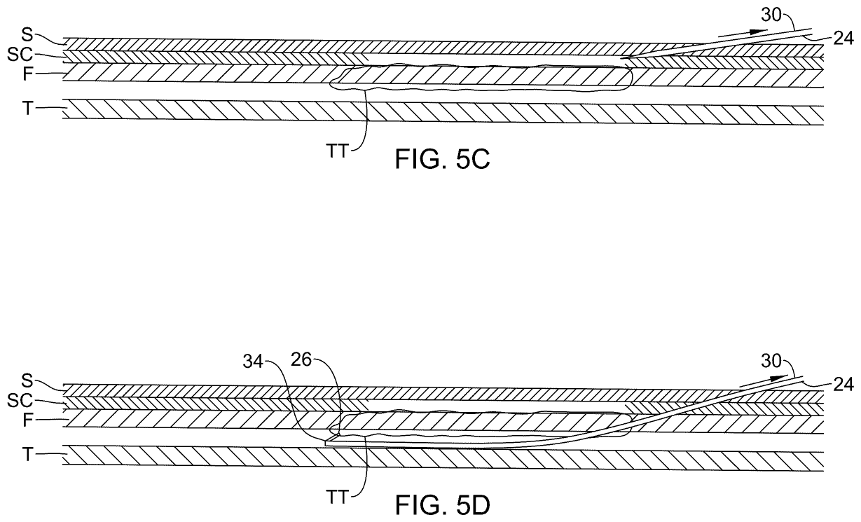

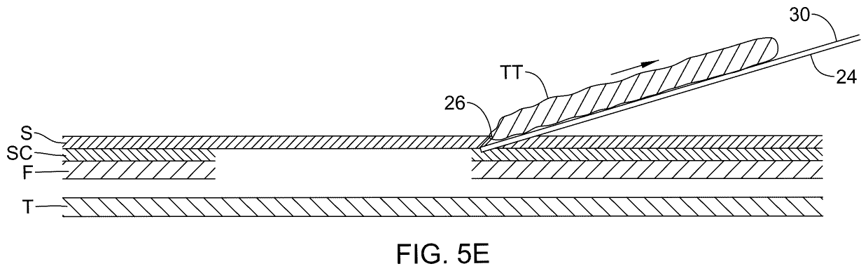

FIGS. 5A-5E illustrate an alternative embodiment of a method for treating Dupuytren's contracture, in this example using the treatment device 20 of FIGS. 4A and 4B. In this embodiment, the patient's hand may be prepared in the same or similar manner as described above, and ultrasound may be used during all or any portion of the procedure to track the location of the treatment device 20 and/or anatomical structures in the hand. In the cross-sectional diagrams of FIGS. 5A-5E, the levels of the patient's hand include skin S, subcutaneous tissue SC, fascia F, tendon T, and target tissue TT (e.g., the central cord or other cord).

In this embodiment, the physician forms a proximal transverse incision through the skin S, superficial to the target tissue TT. Skin edges are then retracted to directly visualize the target tissue TT. Next, as illustrated in FIG. 5A, the physician advances (solid-tipped arrow) the sharp distal tip 34 of the shaft 24 of the treatment device 20 superficially to the target tissue TT and deep to the overlying skin S, passing it through the subcutaneous tissue SC. As shown in FIG. 5B, the treatment device 10 is advanced over the target tissue TT, using the sharp distal tip 34 to dissect through the subcutaneous tissue SC and separate the skin S from the target tissue TT. After sufficiently dissecting through the subcutaneous tissue SC, the shaft 24 is withdrawn at least partially, as shown in FIG. 5C. All of these initial steps are performed with the blade protector 30 fully advanced to prevent the blade 26 from cutting tissue. This is helpful for procedural success, since the blade 26 initially faces toward the skin S.

Referring to FIG. 5D, the shaft 24 of the treatment device 20 may next be advanced again into the hand, this time through the fascia and deep to (below) the target tissue TT. When the shaft 24 is in a desired location to treat the target tissue TT, which may be confirmed by ultrasound, the blade protector 30 is retracted, exposing the blade 26 for cutting. As illustrated in FIG. 5E, the shaft 24 may then be retracted out of the hand to cut and pull out the target tissue TT, which can be excised with a cutting device outside of the hand. This process, or any portion thereof, may be repeated until the target tissue TT has been completely excised. This example of the method and device may be generally referred to as an “upward blade” technique and device. One potential advantage of this second embodiment is that the upward facing blade 26 never faces the tendon T, thus not subjecting this important tissue to potential cutting.

Although the foregoing is believed to be a complete and accurate description of embodiments and features, the invention is not limited to any of the examples or embodiments described herein.

What is claimed is:

1. A device for treating Dupuytren's contracture in a hand of a patient, the device comprising:
a handle;
a shaft extending from the handle and sized to fit through a small incision on the hand and under a target tissue, wherein the shaft comprises a sharp distal tip configured to cut through soft tissue;
a retrograde blade on the shaft, facing proximally toward the handle; and
an opening in the shaft, adjacent the retrograde blade, configured to allow cut tissue to pass therethrough.

2. The device of claim 1, further comprising a tendon guard on at least a portion of the shaft.

3. The device of claim 1, wherein the shaft comprises a cross-sectional shape selected from the group consisting of tubular and semi-tubular.

4. The device of claim 1, wherein the device is a one-piece construction made of one material.

5. The device of claim 1, wherein the handle and the shaft are two separate pieces, and wherein the shaft is removable from the shaft and replaceable with a replacement shaft.

6. The device of claim 1, wherein the retrograde blade faces upward from the shaft.

7. The device of claim 1, wherein the retrograde blade faces downward from the shaft.

8. The device of claim 1, further comprising:
a blade protector slidably disposed along the shaft from the handle to the retrograde blade; and
a slider on the handle, connected to the blade protector, to slide the blade protector along the shaft from a blade protecting position to a blade exposed position.

9. The device of claim 8, wherein the handle and the slider are made of a plastic material, and the shaft, the retrograde blade, and the blade protector are made of one or more metal materials.

10. A method for treating Dupuytren's contracture in a hand of a patient, the method comprising:
advancing a shaft of a treatment device through an incision on the hand, superficial to a target tissue in the hand and deep to skin of the hand;
advancing the shaft to dissect the skin away from the target tissue, using a sharp distal tip of the shaft;
sliding a blade protector of the treatment device proximally to expose a retrograde blade facing downward on the shaft;
pulling proximally on a handle attached to the shaft of the treatment device to cut the target tissue with the retrograde blade; and
pulling the handle further proximally to pull at least a cut portion of the target tissue out of the hand through the incision.

11. The method of claim 10, further comprising:
cutting off the cut portion of the target tissue with a cutting device; and
removing the treatment device from the hand.

12. The method of claim 10, further comprising visualizing at least part of the method using an ultrasound device located outside of the patient.

13. The method of claim 10, further comprising:
advancing the shaft back into the hand; and
using the retrograde blade to remove additional target tissue from the hand.

14. The method of claim 10, wherein advancing the shaft to dissect the skin away from the target tissue comprises moving the shaft in a side-to-side motion to advance the sharp distal tip of the shaft through subcutaneous tissue.

15. The method of claim 10, wherein pulling proximally on the handle attached to and cutting the target tissue causes at least some of the cut portion to pass through an opening in the shaft adjacent the retrograde blade.

16. The method of claim 10, further comprising protecting a tendon in the hand with a tendon guard on the treatment device.

17. The method of claim 10, wherein the target tissue comprises at least one of a cord or fascia.

18. A method for treating Dupuytren's contracture in a hand of a patient, the method comprising:
advancing a shaft of a treatment device through an incision on the hand, superficial to a target tissue in the hand and deep to skin of the hand;
advancing the shaft to dissect the skin away from the target tissue, using a sharp distal tip of the shaft;
repositioning the shaft to a position deep to the target tissue;
sliding a blade protector of the treatment device proximally to expose a retrograde blade facing upward on the shaft;
pulling proximally on a handle attached to the shaft of the treatment device to cut the target tissue with the retrograde blade; and
pulling the handle further proximally to pull at least a cut portion of the target tissue out of the hand through the incision.

19. The method of claim 18, further comprising:
cutting off the cut portion of the target tissue with a cutting device; and
removing the treatment device from the hand.

20. The method of claim 18, further comprising visualizing at least part of the method using an ultrasound device located outside of the patient.

21. The method of claim 18, further comprising:
advancing the shaft back into the hand; and
using the retrograde blade to remove additional target tissue from the hand.

22. The method of claim 18, wherein advancing the shaft to dissect the skin away from the target tissue comprises moving the shaft in a side-to-side motion to advance the sharp distal tip of the shaft through subcutaneous tissue.

23. The method of claim 18, wherein pulling proximally on the handle attached to and cutting the target tissue causes at least some of the cut portion to pass through an opening in the shaft adjacent the retrograde blade.
